(19) United States
(12) Patent Application Publication
(10) Pub. No.: US 2019/0228678 A1
(43) Pub. Date: Jul. 25, 2019

(54) SYSTEM FOR CONTROL OVER FOOD AND DIET AND RELATED METHOD TO REDUCE ENVIRONMENTAL IMPACT

(71) Applicant: Rosemary Elizabeth OSTFELD, East Lyme, CT (US)

(72) Inventor: Rosemary Elizabeth OSTFELD, East Lyme, CT (US)

(21) Appl. No.: 16/242,789

(22) Filed: Jan. 8, 2019

Related U.S. Application Data
(60) Provisional application No. 62/615,394, filed on Jan. 9, 2018.

(51) Int. Cl. G09B 19/00; G06Q 30/06; G16H 20/60

(52) U.S. Cl. CPC G09B 19/0092; G16H 20/60; G06Q 30/0603

(57) ABSTRACT

A system to control a user's food and diet to reduce their environmental impact including a device selected from the group consisting of a desktop computer, laptop, personal digital assistant (PDA), portable computer, tablet PC and wearable computer, storing a software program having instructions causing the device to receive a barcode, image, audio or text data of a food to be consumed by the user; for identifying the food and its ingredients; for analyzing the cost, nutritional content and environmental footprint of said food by a query to a database that stores information about the food; to determine the equivalency of the environmental footprint of the food; to compile each food item consumed by the user to create the user's current diet; to compare the user's current diet to an environmentally sustainable guideline diet; to generate a food recommendation to the user to reduce their environmental impact; and to connect a user to local sustainable food producers.

This application claims the benefit of U.S. provisional application Ser. No. 62/615,394 filed Jan. 9, 2018 which is incorporated herein in its entirety by reference.

### FIELD OF THE INVENTION

The present invention relates to a system for a person to control their food and diet to eat sustainable foods which therefore reduce their environmental impact. The system and methods used analyze the cost, health and nutrition-related information, and the environmental impact of a person's foods and diet choices and providing recommended food items to the person to consume a healthier diet and to reduce their environmental impact. These science-based recommendations are personalized according to a person's economic, health, or environmental sustainability needs or specifications. A person may use the invention system and methods to connect to other individuals as well as local healthy and environmentally sustainable food producers.

### BACKGROUND OF THE INVENTION

It is well known that a poor diet is a leading cause of early death around the world. People fail to consume diets that align with science-based dietary recommendations developed by experts, such as those recommended by the World Health Organization, or published by national governments. Poor diets are also leading to a rise in non-communicable diseases such as cardiovascular disease, type 2 diabetes, hypertension and certain types of cancers.

Simultaneously, agriculture and food systems require numerous natural resources as inputs and have a myriad of impacts on the environment. These inputs and impacts can be quantified. For example, lifecycle inventory analyses (also known as lifecycle inventory assessments) can quantify inputs such as but not limited to land, water, and energy, and impacts such as but not limited to those on soil, biodiversity, water systems, land, and the atmosphere. Food production is also a major emitter of greenhouse gas emissions, and widescale dietary change will be necessary mitigate global climate change and feed a global population of 10 billion.

Research has found that certain diets are better for health and have a smaller impact on the environment. However, people struggle to make food and diet choices that are beneficial for their health and the environment. This can occur due to many reasons including but not limited to lack of knowledge or awareness, and lack of access to affordable, nutritious, and environmentally sustainable food. In particular, cost is a major factor in the food and dietary choices people make.

Therefore, there is a strong need to help people easily track the costs, nutritional content and environmental footprint of the food they consume, as well as provide recommendations.

There is also a need to personalize these recommendations according to a person's economic, health, and environmental sustainability needs or specifications. These recommendations can help people achieve a nutritious and environmentally sustainable diet within their economic means.

Currently there are apps and online platforms that help people track the nutritional content of their food, i.e. My Fitness Pal.

Other representative prior art related to nutritional tracking, food images, and meal planning include the following:

U.S. Patent Publication No. 2010/0173269 to Puri et. al. discloses a method and system for analyzing at least one food item on a food plate. A plurality of images of the food plate is received by an image capturing device. A description of the food item on the food plate is received by a recognition device. The description is at least one of an audio and a text description. At least one processor extracts a list of food items from the description; classifies and segments the at least one food item from the list using color and texture features derived from the plurality of images; and estimates the volume of the classified and segmented at least one food item. The processor is also configured to estimate the caloric content of the at least one food item.

U.S. Patent Publications No. 2011/0009708 and 2014/0287384 both to Boyes disclose systems having an incorporated software program attempts to tailor a person's diet with the person's desired nutritional goal and their current food consumption.

U.S. Pat. No. 5,841,115 to Shepley discloses a method for providing personalized nutrition information to an individual comprising the following steps: (a) inputting personal data relating to an individual; (b) inputting data identifying at least one food product which the individual intends to purchase or consume; (c) accessing prestored information relating to the at least one food product which the individual intends to purchase or consume; (d) generating and outputting information about the at least one food product which the individual intends to purchase or consume pertinent to the input personal data.

U.S. Patent Publication No. 2016/0071431 to Jain disclose apparatuses, methods and storage media associated with standardization or normalization of a food description. In embodiments, an apparatus may include one or more processors and a food data pre-processing engine. The food data processing engine may, when operated by the one or more processors, cause the apparatus to receive a description of a food item, process the description to generate a standardized or normalized description for the food item, and store the standardized or normalized description in a food record in a food data storage arrangement of a fitness management system.

U.S. Patent Publication No. 2016/0103910 to Kim discloses apparatuses, methods and storage media associated with food item categorization. In embodiments, an apparatus may include one or more processors and a food categorization engine. The food categorization engine may be operated by the one or more processors to cause the apparatus to receive a description of a food item; analyze the description of the food item; and select one or more food categories with which to associate the food item based at least in part on a result of the analysis.

U.S. Patent Publication No. US2012/0083669 to Abujbara discloses personal nutrition and wellness advisor system comprised of at least one computing platform, medical and nutrition knowledge databases, food nutrient databases, receives and analyzes each user's initial personal and health-related information to intelligently estimate his initial energy & nutrient budgets and physical activity needs; interactively constructs and presents to the user personalized energy and nutrient content-based, location-based, activity-based, and event-based ranked available food item lists and recipes to encourage the user to be physically active and to consume food items containing the most needed nutrients; tracks the user food consumption, physical activity, updates to energy and nutrient budget balances, any physiological parameter measurements, any taken medication types, doses, and times, and any other external events which may cause changes to user-specific health-related variables; and automatically varies the types, rankings, and/or portions of the food items in the recommended available food item list based on the results of tracked activities.

U.S. Pat. No. 9,779,442 to Cimic et al. provides a method for identifying garden items and for recommending items to the user.

There is scientific evidence that food and diet choice impact both health and the environment. None of the references above provide a system and method to provide the user with the ability to control their food and diet to achieve a healthy, affordable and environmentally sustainable diet. However, the present invention provides an advantage over prior practice by incorporating an environmental sustainability component. Accordingly, a main object of the invention is to provide a system to help people achieve a healthy, affordable, and environmentally sustainable diet.

Another object of the invention is to allow people to enter specifications related to (1) Cost, i.e. how much would the person wish to spend on food per week; (2) Health, i.e. low sugar, low glycemic index, gluten free, lactose free; (3) Environmental impact, i.e. greenhouse gas emissions, land, water, biodiversity, soil; and (4) Diet preference, i.e. science-based dietary guidelines, or pescatarian, vegetarian, vegan diets.

Yet another object of the invention system is to include an image, scan a barcode, provide a text or audio description of a food.

Another object of the invention is to Identify the cost, nutritional content (i.e. vitamins, minerals, macro-nutrients), and environmental impact (i.e. greenhouse gas emissions, land, water, biodiversity, soil) of food and ingredients to aid individuals in making healthier and more environmentally sustainable food choices.

Another object of the invention is to provide equivalencies to help people understand environmental impacts. For instance, 1 tonne food-related greenhouse gas emissions is equivalent to approximately the greenhouse gas emissions associated with a flight from London to New York.

Yet another object of the invention is to provide a recommendation for sustainable food including meal plans with recipes and shopping lists considering personal specifications.

Another object of the invention is to provide a platform for users including people and food producers such as farms, retailers and food manufacturers such that they can engage with one another about healthy and environmentally sustainable foods.

### SUMMARY OF THE INVENTION

In the present invention, these purposes, as well as others which will be apparent, are achieved generally by providing a system for a person to control their food and diet and enables them to eat sustainable foods which therefore reduce their environmental impact.

The system includes a device selected from the group consisting of a desktop computer, laptop, personal digital assistant (PDA), portable computer, tablet PC and wearable computer

The system device stores a software program having instructions to implement a method for identifying a food, analyzing the cost, health and nutrition-related information, and environmental impact of a person's food and diet choices and providing recommendations to the person to consume a healthier diet and to reduce their environmental impact.

The system includes a method for inputting economic, health, or environmental impact related needs or specifications.

The system includes a method for identifying a food, analyzing the cost, nutritional content, and environmental footprint of a food.

A person uses a device to scan a barcode or take an image of a food, or input data for a specific food. The food and ingredients are identified by the system.

Data from the device related to a particular food is sent to a server where the cost, nutritional content and environmental footprint are analyzed. The user is provided with the cost, nutritional content, and environmental footprint data. This method allows a person to track changes in spending, dietary behavior, and their own food-related environmental footprint.

Recommendations for food using the invention system is provided to the user.

A method for engaging with other people and sustainable food producers is also provided.

Other objects, features and advantages of the present invention will be apparent when the detailed description of the preferred embodiments of the invention are considered with reference to the drawings, which should be construed in an illustrative and not a limiting sense.

### BRIEF DESCRIPTION OF THE DRAWINGS

FIG. 1 illustrates an example according to the invention system of entering specifications related to the user's personal health;

FIG. 2 illustrates an example according to the invention system of entering specifications related to the user's dietary habits;

FIG. 3 illustrates an example according to the invention system of entering specifications related to the user's environment;

FIG. 4 illustrates an example according to the invention system of entering specifications related to the user's food-related spending;

FIG. 5 is a schematic illustration of the invention system user specified information components related to personal health, dietary habits, environment, and food-related spending to produce personalized health and environmentally sustainable food and diet recommendations;

FIG. 6 is a schematic illustration of an example according to the invention system of providing food and diet-related recommendations and shopping lists for environmentally sustainable food products;

FIG. 7 illustrates the system according to the invention of obtaining a barcode, image, text or audio description of food items to identify a particular food;

FIGS. 8 and 9 are schematic illustrations of the invention system analyzing the cost, health-related information, and environmental footprint of a particular food item;

FIG. 10 illustrates an example according to the invention of providing environmental equivalencies of a food item of the user's diet;

FIG. 11 illustrates an example according to the invention system of comparing the cost, health-related information, and environmental impact of the diet of the user to the general population;

FIG. 12 is a schematic illustration of the invention system of users engaging with one another, and/or local farms, and/or sustainable food producers;

FIG. 13 illustrates an example of the sustainable food platform according to the invention system where a user engages with other entities including other people, and/or environmentally sustainable food producers such as farms, retailers, and/or food manufacturers;

FIG. 14 illustrates an example according to the invention system of information provided to the user about a sustainable food producer.

### DETAILED DESCRIPTION OF THE INVENTION

In accordance with the present invention, a system is provided for a user to control their food and diet to reduce their environmental impact by making scientifically based recommendations according to the user's economic, health and environmentally sustainable needs or specifications.

In particular, the invention system to control a user's food and diet to reduce their environmental impact utilizes a device selected from the group consisting of a desktop computer, laptop, personal digital assistant (PDA), portable computer, tablet PC and wearable computer.

In general, the device stores a software program that enables a user to specify information related to their personal health, dietary patterns, food-related spending, and environmental impact information.

Specifically, the device stores a software program having instructions causing the device to receive a barcode, image, audio or text data of a food to be consumed by the user.

The device also stores a software program having instructions for identifying said food and its ingredient.

The device also stores a software program having instructions for analyzing the cost, nutritional content and environmental footprint of said food by a query to a database that stores information about the food.

The device also stores a software program having instructions to determine the equivalency of the environmental footprint of the food.

The device also stores a software program having instructions to compile each food item consumed by the user to create the user's current diet.

The device also stores a software program having instructions to compare the user's current diet to an environmentally sustainable guideline diet.

The device also stores a software program having instructions to generate food and diet recommendations to the user to reduce their environmental impact.

The device also stores a software program having instructions to compare the user's current diet to the diet of other users.

The device also stores a software program having instructions to generate recommendations for individual foods, meal plans and shopping lists for the user to reduce their environmental impact and other environmental-related values.

The device also stores a software program having instructions to analyze a food and assign said food a greenhouse gas emissions value.

The device also stores a software program having instructions to analyze a food and assign said food environmental impact values based on environmental impact data including land, water, soil, biodiversity and chemical impacts.

The device also stores a software program having instructions to engage users with other users on environmentally sustainable food and diets.

The device also stores a software program having instructions to connect and find products available from local sustainable food producers such as farms.

The device also stores a software program having instructions to connect and receive information on local sustainable food producer including their production methods, foods that are produced, the environmental impacts of said foods, and the ability to purchase said foods.

The device also stores a software program having instructions to allow users to connect with and engage sustainable food producers including retailers and manufacturers, to learn about the environmental impact of products.

The invention also includes a method to reduce a user's environmental impact by control over food and diet.

The invention method uses a device selected from the group consisting of a desktop computer, laptop, personal digital assistant (PDA), portable computer, tablet PC and wearable computer. The device stores a software program with instructions related to the user's personal health, dietary patterns, food-related spending, and environmental impact information in said device.

The method steps include causing the device to receive a barcode, image, audio or text data of a food to be consumed by the user; identifying said food and its ingredients; analyzing the cost, nutritional content and environmental footprint of said food by a query to a database that stores information about the food; determining the equivalency of the environmental footprint of the food; compiling each food item consumed by the user to create the user's current diet; comparing the user's current diet to an environmentally sustainable guideline diet; and generating food and diet recommendations to the user to reduce their environmental impact.

Additional method steps include the step of comparing the user's current diet to the diet of other users; generating recommendations for individual foods, meal plans and shopping lists for the user to reduce their environmental impact and other environmental-related values; analyzing a food and assigning said food a greenhouse gas emissions value and/or food environmental impact values based on environmental impact data including land, water, soil, biodiversity and chemical impacts; engaging users with other users on environmentally sustainable food and diets; connecting and finding products available from local sustainable food producers such as farms; connecting and receiving information on local sustainable food producer including their production methods, foods that are produced, the environmental impacts of said foods, and the ability to purchase said foods; and connecting with and engaging sustainable food producers including retailers and manufacturers, to learn about the environmental impact of products.

The invention embodiments are described below in reference to the related drawings.

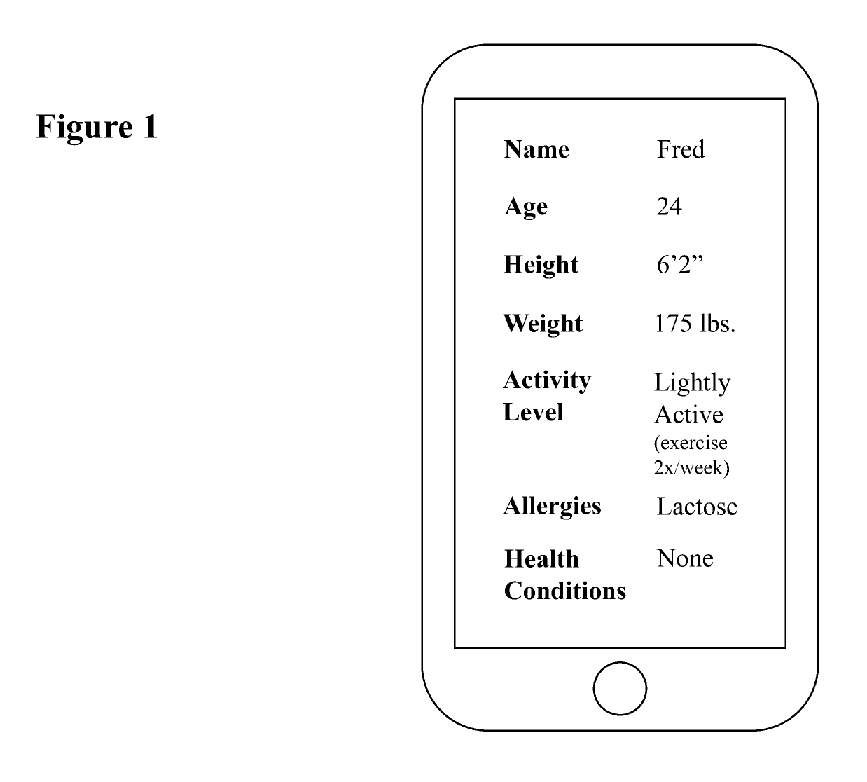

As shown in FIG. 1 user specifications related to personal health are illustrated. These specifications include basic physical characteristics such as the user's height, weight, age, and activity level. It also includes medical conditions such as food allergies i.e. allergies to soy, gluten, nuts, lactose; and/or current medical conditions i.e. Type 2 diabetes, hypertension, etc.

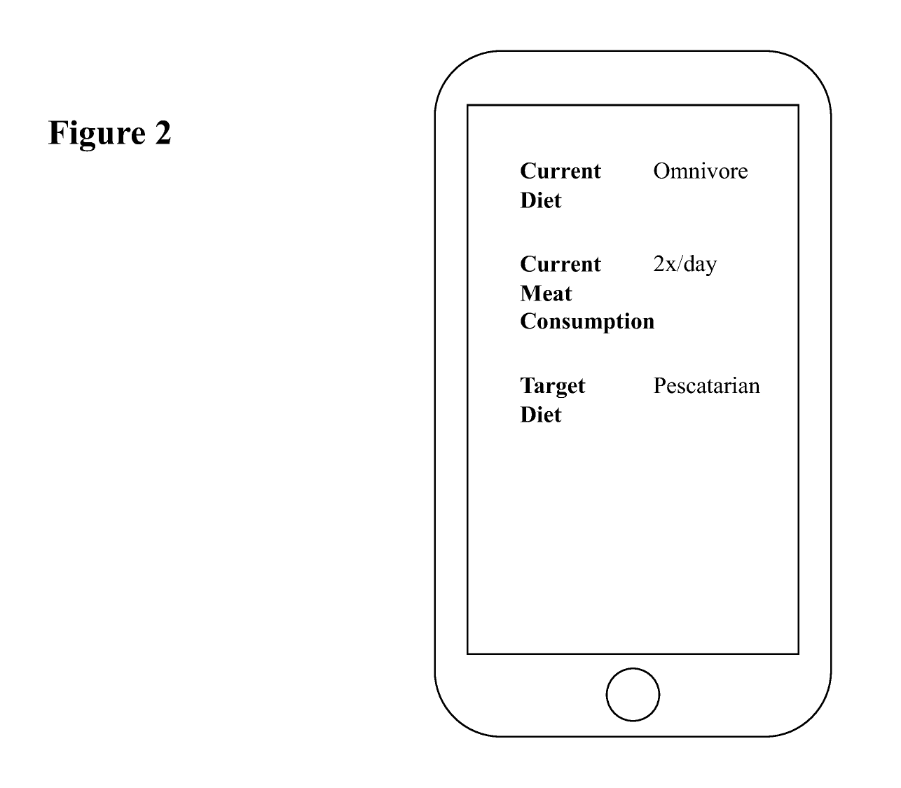

FIG. 2 illustrates the user specifications related to the user's dietary habits. These specifications include current dietary patterns. For example, the general type of diet, the times per week the user eats meat, etc. It could also include the user's target dietary patterns i.e. science-based dietary guidelines, or diet type such as low-meat, pescatarian, vegetarian, vegan.

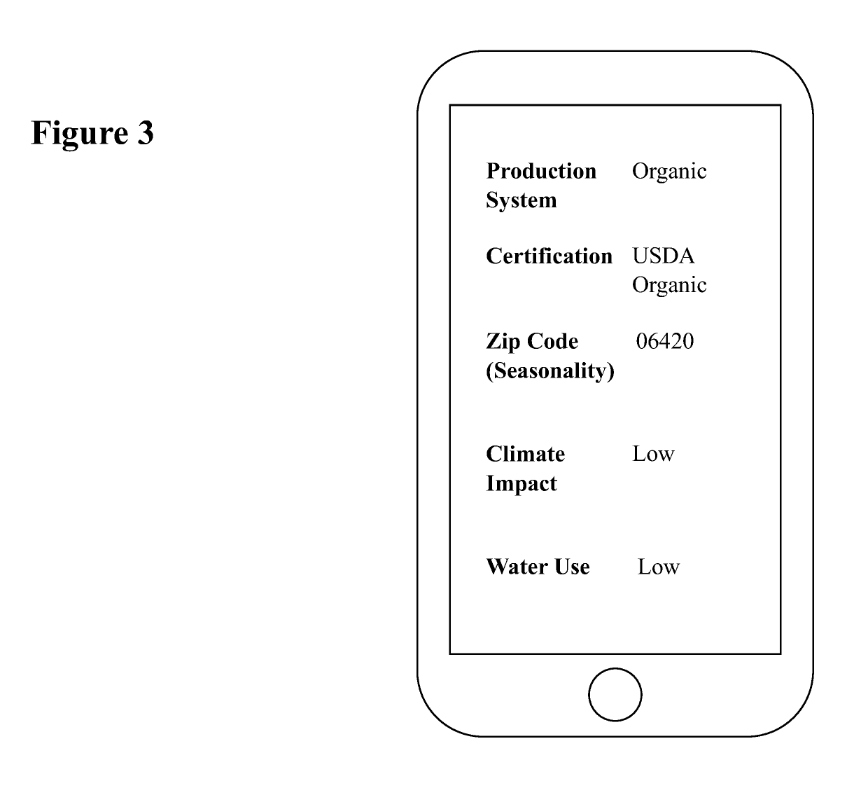

FIG. 3 illustrates the user specifications related to the environment. These specifications include factors related to food production such as non-GMO, organic, regenerative organic, conventional; environmental certifications i.e. USDA organic; food seasonality based on the user's location (i.e. is the food currently in season); greenhouse gas emissions; water and land use (i.e. previous land cover—was the land recently deforested?), soil quality, biodiversity, and chemical inputs (i.e. chemical fertilizers, chemical pesticides). Based on the production type selected in FIG. 3 (i.e. organic) potential impacts on soil, and chemical inputs could be determined. Organically produced foods are known to have a lower impact on the environment in terms of effects on land and water due to fewer chemical inputs.

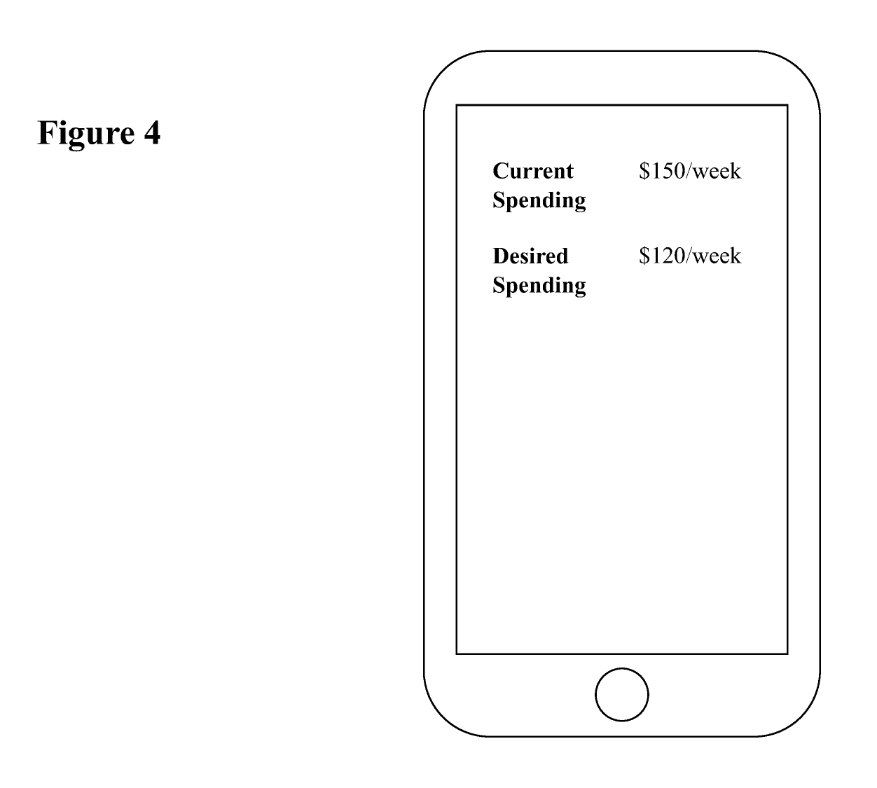

FIG. 4 illustrates user specifications for food-related spending. These specifications include current spending patterns, and target spending levels. For example, if the user does not want to spend more than $150 per week on food, they can input the desired spending level to stay within a lower budget such as $120 per week.

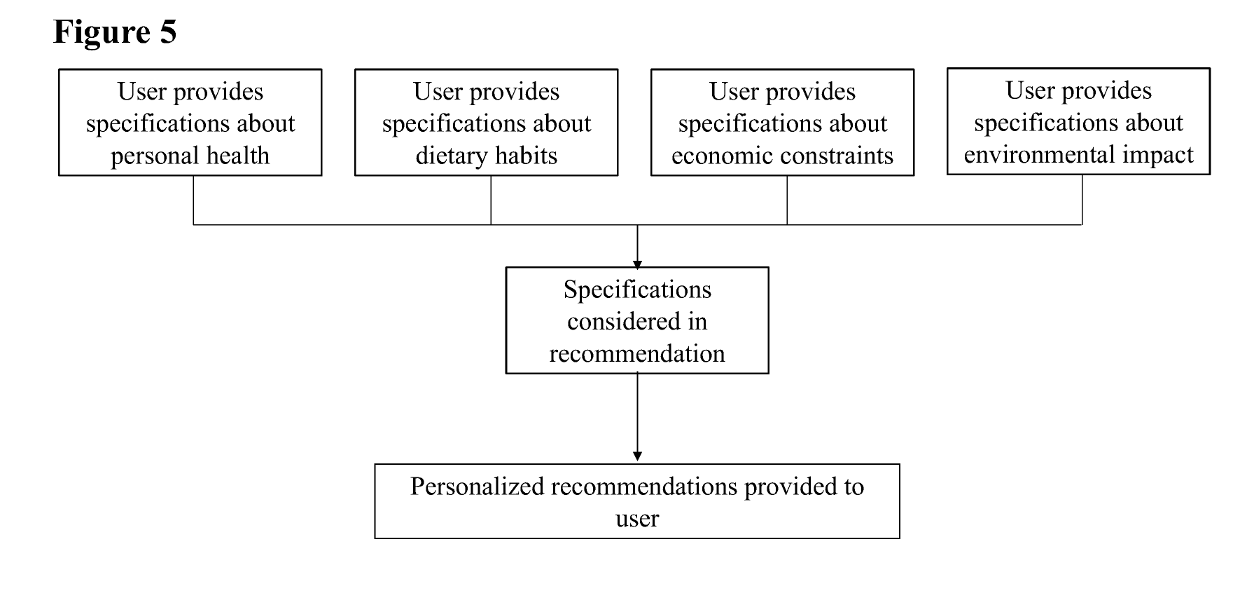

As shown in FIG. 5 user specified data is input in to the invention system relating to personal health, dietary habits, environment, and food-related spending to produce food and diet recommendations for the user. The output to the user is a personalized health and environmentally sustainable diet recommendations.

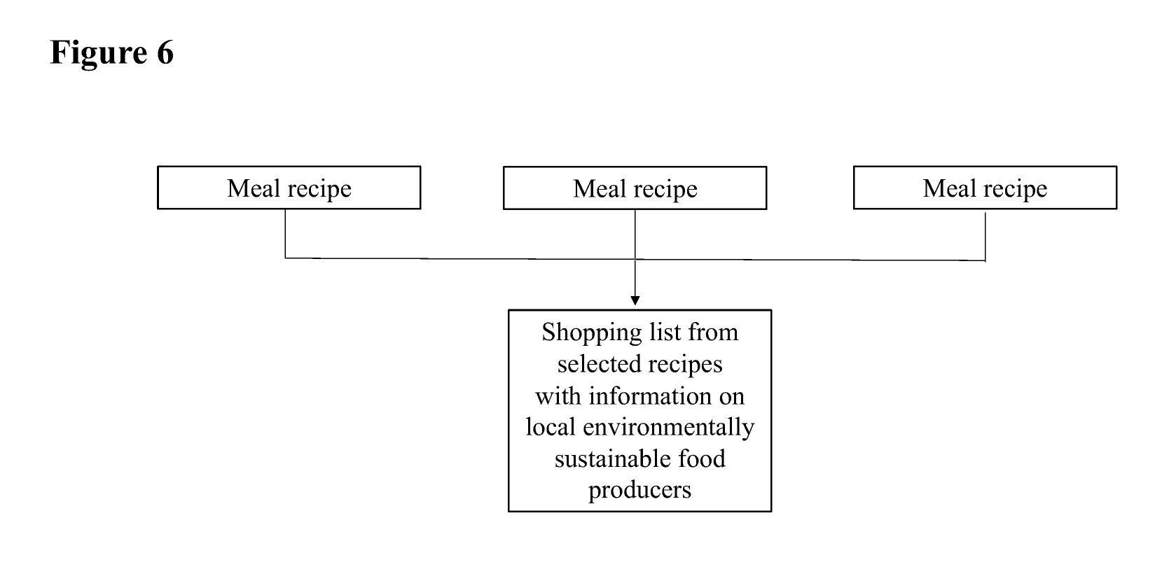

FIG. 6 illustrates an example of providing food and diet-related recommendations, such as meal plans with recipes and shopping lists, produced using an algorithm based on the analysis of the user specified information and also considering science-based dietary guidelines so the person has the correct nutrients and is minimizing their environmental impact.

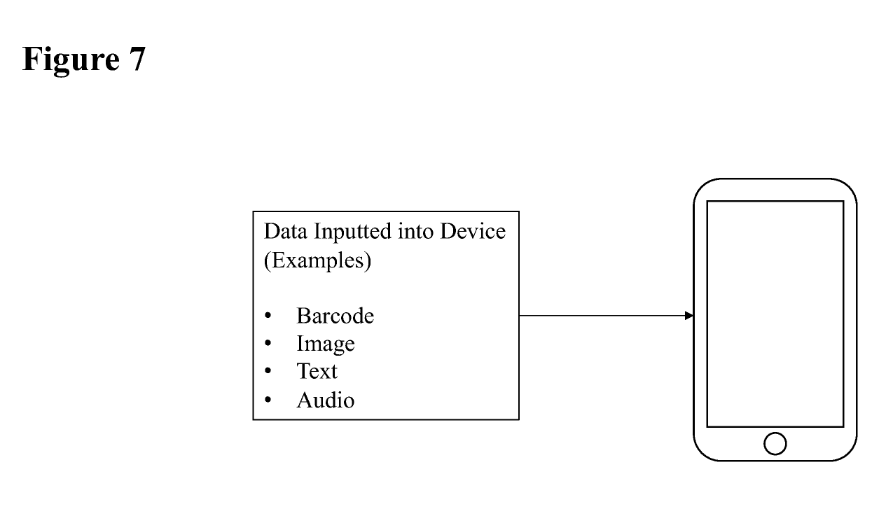

FIG. 7 illustrates obtaining a barcode, image, text or audio description of food items to identify that food. The person inputs a food item into the invention system by scanning the barcode using a device, taking an image, using text or providing an audio description.

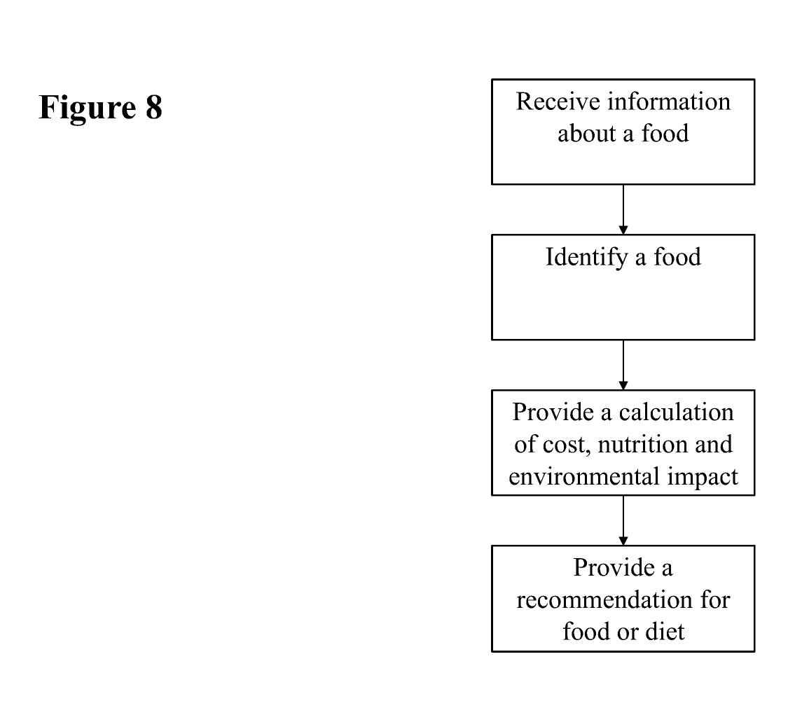

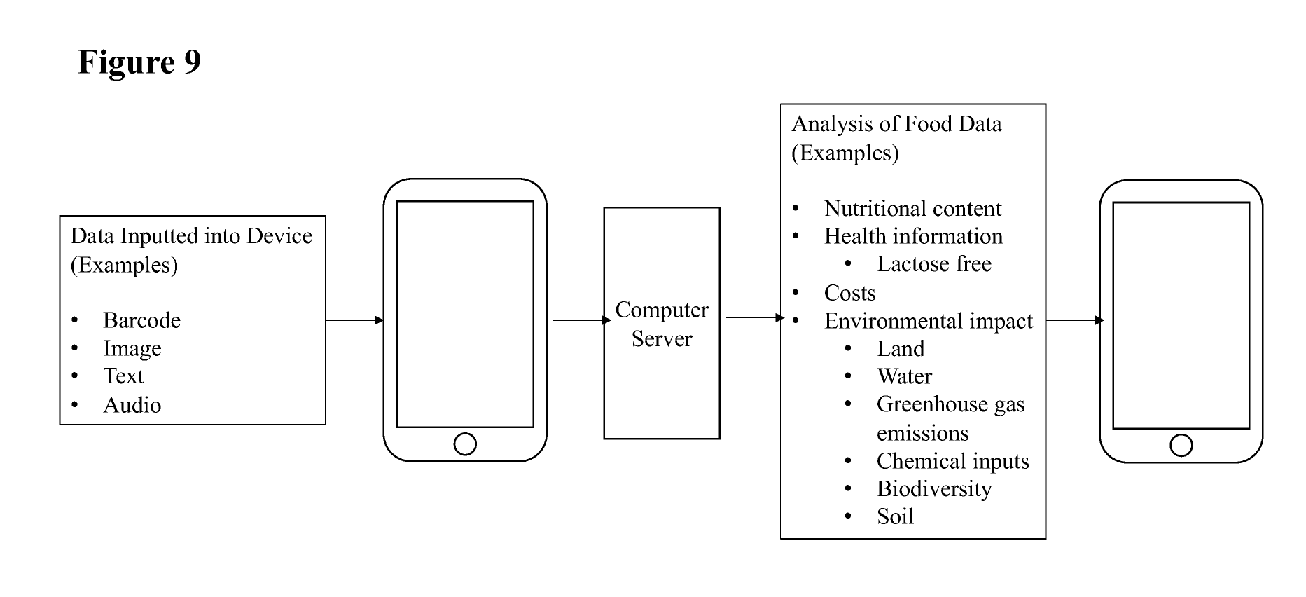

As illustrated in the schematic of FIG. 8 and FIG. 9, each food item data as entered above, is analyzed for its cost, nutritional and health-related information, and the environmental impact of a food item.

After a food is retrieved using a device as described in potential embodiments illustrated in FIG. 7, the food is identified. After the food and/or ingredients is/are identified, the cost, nutritional content, and environmental footprint of a food is presented to the person. For example, nutrition information such as calories, fat, protein, carbohydrates, vitamins and minerals may be provided.

The environmental footprint information such as greenhouse gas emissions, water use required to produce the food, energy use required to produce the food, land use required to produce the food, impacts on biodiversity, impacts on soil, and pollution from producing the food are provided. This information will be calculated based on existing life-cycle analysis data, or be provided by the food producer, similar to the way nutritional information is currently portrayed on food packaging.

As new items are added over a specific timeframe—daily, weekly or monthly—a recommendation may change based on the user's specifications. To illustrate this example more clearly: A person may have specified that they are trying to reduce their food-related greenhouse gas emissions. Science-based dietary recommendations may specify that a person should only eat two servings of a certain food product per week to have an optimally healthy and environmentally sustainable diet. After the person inputs that they have eaten one serving of that food product in a given week, the system may adjust the recommendation so that it is recommended the person only eats one more serving of that food product during that week. The person may also be recommended to consume other food items, such as fruits and vegetables in the upcoming meals to have a more nutritionally balanced and environmentally sustainable diet.

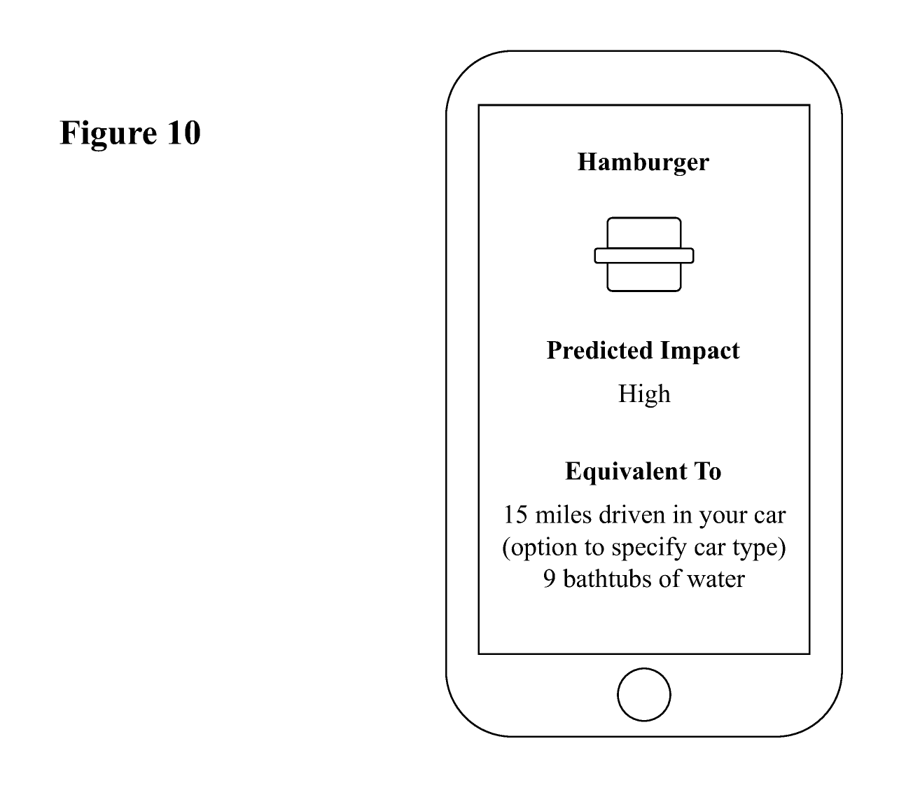

FIG. 10 illustrates an example of providing environmental equivalencies of a food item of the user's diet. The invention utilizes a proprietary formula for calculating and assigning an equivalency value to each food item and its production method chosen by the user. The quantities used to convey greenhouse gas emissions, for example, are often complex and difficult for the average person to understand. To make these quantities more comprehendible for the average person, they may be conveyed in terms of equivalencies. For example, one metric tonne of greenhouse gas emissions may be conveyed as being equivalent to approximately taking a transatlantic flight from New York City to London.

Equivalencies for land, water, greenhouse gas emissions, and energy are provided for the user's food choices or diet. For example, data exists on the greenhouse gas emissions associated with flying, driving different types of vehicles, leaving a lightbulb plugged in etc. Information from a database containing such information is accessed by the system and an algorithm is used to calculate the equivalency for food items. For example, if a person eats 0.5 kilograms of beef this is equal to 33.8 kilograms of GHG emissions. An average passenger vehicle emits 411 grams GHG emissions per mile driven. Therefore, eating 0.5 kilograms of beef is equivalent to driving approximately 82 miles in the average passenger vehicle.

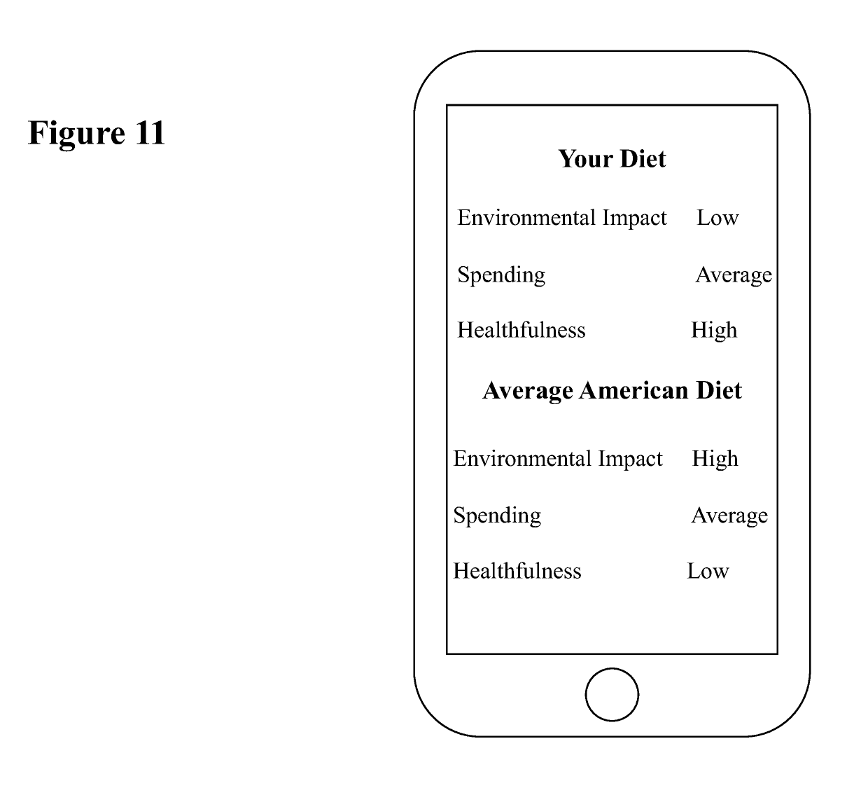

FIG. 11 illustrates an example of comparing the cost, health-related information, and environmental impact of the diet of the user to the general population. This information allows the user to assess their progress towards achieving a healthy and environmentally sustainable diet.

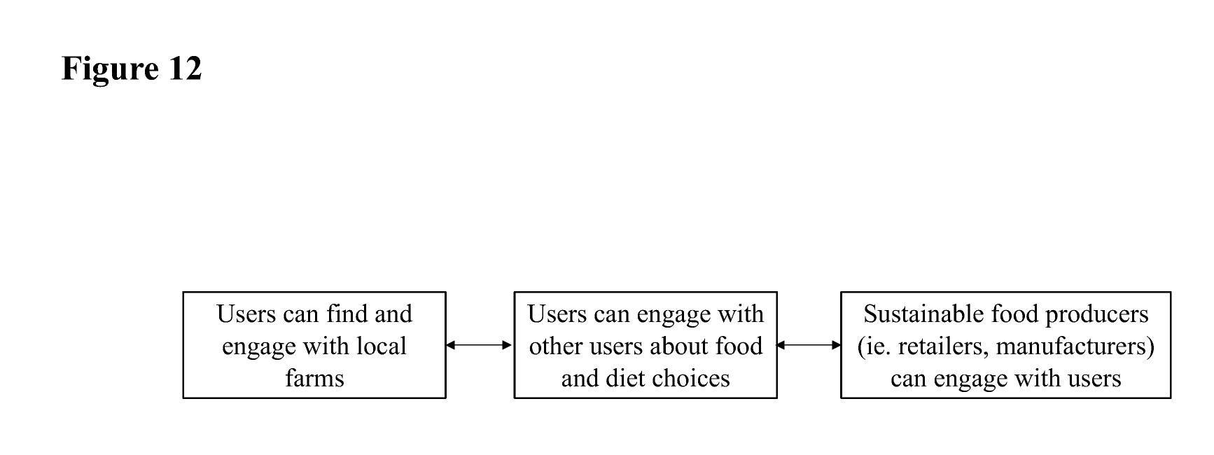

FIG. 12 is a schematic illustration of the invention system of users engaging with one another, and/or local farms, and/or sustainable food producers.

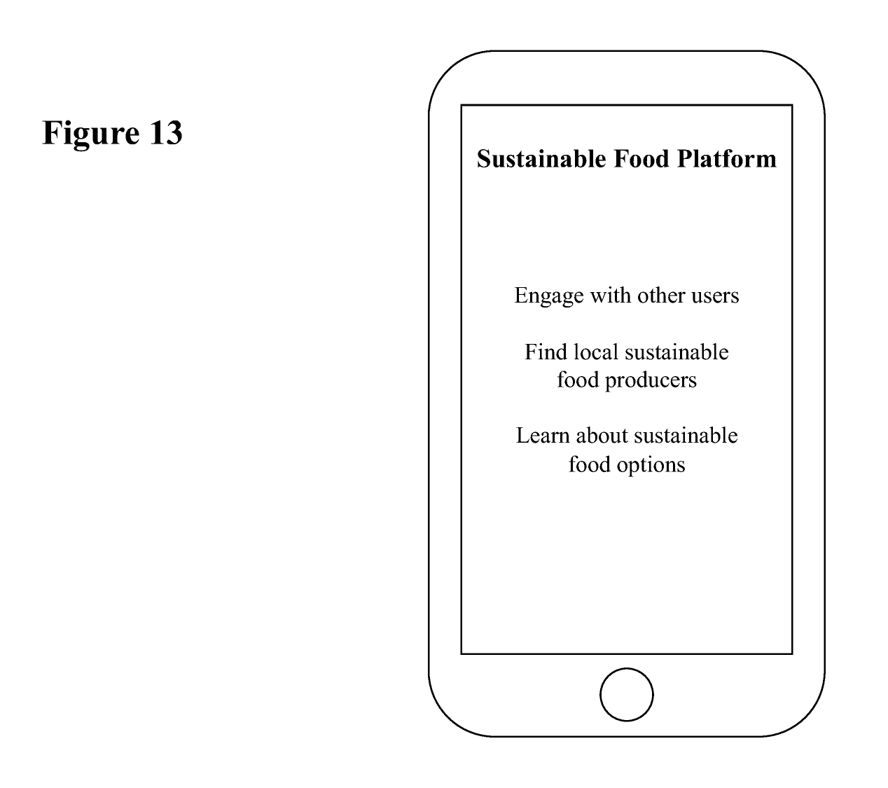

FIG. 13 illustrates an example of a user of the invention system engaging with other entities including other people, and/or environmentally sustainable food producers such as farms, retailers, and/or food manufacturers. A sustainable food manufacturer may use this method to build awareness of their product. The user may compete with other people to complete challenges related to food, i.e. reducing food-related greenhouse gas emissions, or sourcing food from a local farm.

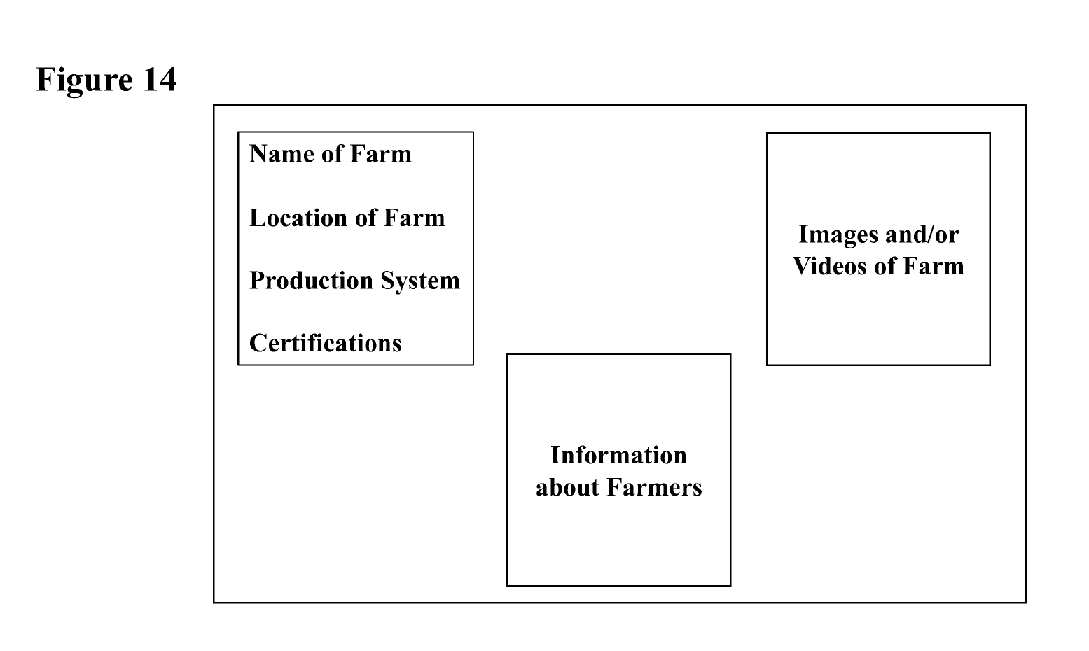

FIG. 14 illustrates an example of information provided to the user about a sustainable food producer. For example, if the sustainable food producer was a farm, the information could include the farm's name and location, information about the farmers who produced the food, and images and/or videos of the farm. An aim of the invention is to encourage people to see where their food comes from and how it is produced to increase traceability and transparency within the agriculture and food supply chain.

The foregoing description of various and preferred embodiments of the present invention has been provided for purposes of illustration only, and it is understood that numerous modifications, variations and alterations may be made without departing from the scope and spirit of the invention as set forth in the following claims

What is claimed is:

1. A system to control a user's food and diet to reduce their environmental impact, said system comprising:
a device selected from the group consisting of a desktop computer, laptop, personal digital assistant (PDA), portable computer, tablet PC and wearable computer;
said device stores a software program having instructions causing the device to receive a barcode, image, audio or text data of a food to be consumed by the user;
said device stores a software program having instructions for identifying said food and its ingredients;
said device stores a software program having instructions for analyzing the cost, nutritional content and environmental footprint of said food by a query to a database that stores information about the food;
said device stores a software program having instructions to determine the equivalency of the environmental footprint of the food;
said device stores a software program having instructions to compile each food item consumed by the user to create the user's current diet;
said device stores a software program having instructions to compare the user's current diet to an environmentally sustainable guideline diet; and
said device stores a software program having instructions to generate food and diet recommendations to the user to reduce their environmental impact.

2. The system according to claim 1, wherein said device stores a software program that enables a user to specify information related to their personal health, dietary patterns, food-related spending, and environmental impact information.

3. The system according to claim 1, said device stores a software program having instructions to compare the user's current diet to the diet of other users.

4. The system according to claim 1, wherein said device stores a software program having instructions to generate recommendations for individual foods, meal plans and shopping lists for the user to reduce their environmental impact and other environmental-related values.

5. The system according to claim 1, wherein said device stores a software program having instructions to analyze a food and assign said food a greenhouse gas emissions value.

6. The system according to claim 1, wherein said device stores a software program having instructions to engage users with other users on environmentally sustainable food and diets.

7. The system according to claim 1, wherein said device stores a software program having instructions to analyze a food and assign said food environmental impact values based on environmental impact data including land, water, soil, biodiversity and chemical impacts.

8. The system according to claim 1, wherein said device stores a software program having instructions to connect and find products available from local sustainable food producers such as farms.

9. The system according to claim 1, wherein said device stores a software program having instructions to connect and receive information on local sustainable food producer including their production methods, foods that are produced, the environmental impacts of said foods, and the ability to purchase said foods.

10. The system according to claim 1, wherein said device stores a software program having instructions to allow users to connect with and engage sustainable food producers including retailers and manufacturers, to learn about the environmental impact of products.

11. A method to reduce a user's environmental impact by control over food and diet comprising the steps of:
using a device selected from the group consisting of a desktop computer, laptop, personal digital assistant (PDA), portable computer, tablet PC and wearable computer, wherein said device stores a software program with instructions related to the user's personal health, dietary patterns, food-related spending, and environmental impact information in said device;
causing the device to receive a barcode, image, audio or text data of a food to be consumed by the user;
identifying said food and its ingredients;
analyzing the cost, nutritional content and environmental footprint of said food by a query to a database that stores information about the food;
determining the equivalency of the environmental footprint of the food;
compiling each food item consumed by the user to create the user's current diet;
comparing the user's current diet to an environmentally sustainable guideline diet; and
generating food and diet recommendations to the user to reduce their environmental impact.

12. The method according to claim 11, further comprising the step of comparing the user's current diet to the diet of other users.

13. The method according to claim 11, further comprising the step of generating recommendations for individual foods, meal plans and shopping lists for the user to reduce their environmental impact and other environmental-related values.

14. The method according to claim 11, further comprising the step of analyzing a food and assigning said food a greenhouse gas emissions value and/or food environmental impact values based on environmental impact data including land, water, soil, biodiversity and chemical impacts

15. The method according to claim 11, further comprising the step of engaging users with other users on environmentally sustainable food and diets.

16. The method according to claim 11, further comprising the step of connecting and finding products available from local sustainable food producers such as farms.

17. The method according to claim 11, further comprising the step of connecting and receiving information on local sustainable food producer including their production methods, foods that are produced, the environmental impacts of said foods, and the ability to purchase said foods.

18. The method according to claim 11, further comprising the step of connecting with and engaging sustainable food producers including retailers and manufacturers, to learn about the environmental impact of products.
